(19) United States
(12) Patent Application Publication
(10) Pub. No.: US 2010/0262182 A1
(43) Pub. Date: Oct. 14, 2010

(54) Occlusion Instruments Comprising Bioresorbable Radiopaque Polymeric Materials, As Well As Related Products, Methods And Uses

(75) Inventors: Robert Moszner, Bad Klosterlausnitz (DE); Kathrin Schmidt, Kahla (DE); Norbert Moszner, Liechtenstein (LI); Claudia Rode, Jena (DE); Thomas Pautsch, Paitzdorf (DE); Ralf-Peter Gottlöber, Bad Blankenbury (DE); Matthias Schnabelrauch, Jena (DE)

Correspondence Address: INSKEEP INTELLECTUAL PROPERTY GROUP, INC, 2281 W. 190TH STREET, SUITE 200, TORRANCE, CA, 90504, US

(73) Assignee: OCCLUTECH GmbH

(21) Appl. No.: 12/600,459

(22) PCT Filed: May 15, 2008
(86) PCT No.: PCT/EP2008/055996

§ 371 Date: Jun. 11, 2010

Related U.S. Application Data
(60) Provisional application No. 60/940,607, filed on May 29, 2007.

(30) Foreign Application Priority Data
May 15, 2007 (EP) 07108281.2

(51) Int. Cl. A61B 17/03; D01D 5/08

(52) U.S. Cl. 606/213; 264/210.8

(57) ABSTRACT

Described are occlusion instruments (1, 2, 3, 4) comprising bioresorbable and thermoplastically deformable polymers with or without shape memory characteristics, which contain radiopaque building groups in the repeat units of the polymer chains and/or are modified with bioresorbable, radiopaque nanoparticles. The polymers display sufficient strength and deformability, good bioresorbability and sufficient visibility in radiance and are particularly useful for producing metal-free occlusion instruments for closing defects of the septum in the heart, the positioning of which being monitorable using routine diagnostic methods.

### FIELD OF INVENTION

The present invention pertains to occlusion instruments comprising bioresorbable, radiopaque polymers and their use for producing occlusion instruments used for occluding defects of the septum in the heart and whose positioning is monitorable using X-ray diagnostic methods.

### BACKGROUND

Radiopaque bioresorbable polymers are known from some medical devices such as stents. Radiopaque polymer-based stents are described in WO 2006/022754 (J. B. Kohn et al.). The polymers, based on halogen-containing (Br, I) diphenol building blocks, are bonded together via dicarboxylic acids and/or polyalkylene oxides via ester or carbonate bonds. The diphenols are prepared by reaction of iodated/noniodated tyrosine ethyl or tertbutyl ester, for example tyrosine ethyl ester (TE), with iodated/noniodated deaminotyrosine, for example 3-(3,5-diiodo-4-hydroxyphenyl)propionic acid (3,5-diiododeaminotyrosine: 12DAT), cf. Formula 1.

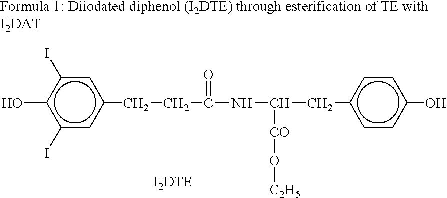

The patent application US 2006/0036316 A1 (J. Zeltinger et al.) discloses linking analogous iodine-containing diphenols via further groups, such as phosphate, phosphonate or iminocarbonate groups, to form radiopaque, bioresorbable polymers.

In the preceding patent references, the radiopaque groups are directly attached on the polymeric backbone chain, which is preparatively costly and inconvenient and distinctly restricts the range of possible variations.

By contrast, the patent application US 2005/0036946 A1 (P. P. Chandrashekhar et al.) discloses radiopaque biodegradable compositions based on synthetic and natural biodegradable polymers modified with iodine-containing end groups. For example, suitably 2-tuply terminated (OH, NH₂) linear polymers, for example poly(caprolactone), cf. Formula 2, poly(lactide) or polyethers, or n-tuply terminated graft, block or star copolymers are end group functionalized with suitable iodine-containing derivatives, for example triiodo-benzoic acid or triiodophenol. The main disadvantage of these radiopaque biodegradable polymers is that only polymers having a low iodine content and hence low radiopaque capacity are so obtainable.

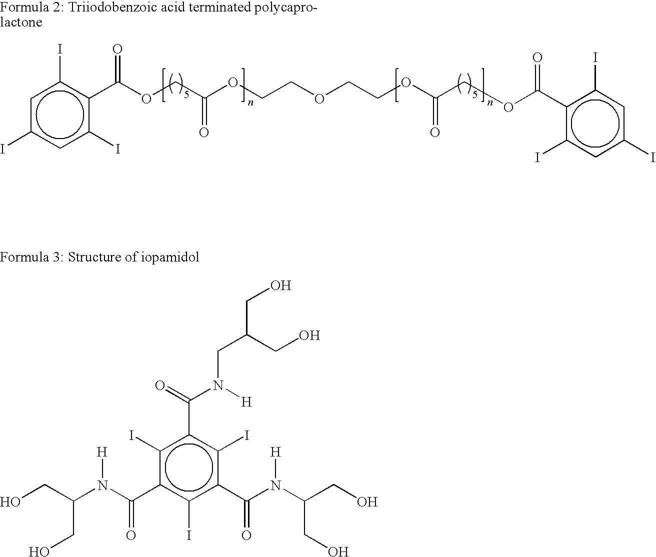

In addition, Chandrashekhar et al. also describe in US 2005/0036946 A1 the crosslinking of biodegradable biopolymers, e.g. albumin, collagen or chitosan, with suitable iodine-containing compounds, e.g. iopamidol, see Formula 3, and also the use of the radiopaque biodegradable compositions for the controlled release of active components. Notable is also, in this way of synthesizing radiopaque biodegradable polymers, the achievable radiopaque capacity limit is, more or less, defined by the usage of high molar mass biopolymers.

A bioresorbable, radiopaque marker for imaging the medical device when using endoprotheses is described in the patent application US 2006/0004440 A1 (J. S. Stinson et al.). The polymer matrix of the markers is based on known bioresorbable polymers, such as poly(L-lactide) or poly(D-lactide), which degrade comparatively slowly, or poly(glycolide) or poly(dioxanone), which degrade comparatively faster. The radiocontrast is obtained through the incorporation of metal particles, e.g. of the elements Ti, Zr, Pt or Au, or of organic compounds containing the elements Br, I, Ba or Bi in bonded form.

The patent application WO 01/85214 A1 describes radiopaque compositions based on polymers or monomers containing non-leachable radiopaque components. The covalent attachment of these radiopaque components is described, the attachment taking the form of linking known radiopaque compounds—including various iodine compounds—with monomer or polymer with suitable functional groups, e.g. isocyanate, ester, aldehyde or epoxide. The polymers used include known synthetic or natural polymers, but not biodegradable or bioresorbable (bioabsorbable) polymers.

The patent application WO 02/089863 A1 describes vaso-occlusive devices and methods based on metal-free materials, i.e., one or more biodegradable polymers. The materials may contain bioactives or radiopaque additives. These comprise known contrast media, particularly metal powders of titanium, gold, tungsten or bismuth and also barium sulfate or gandolonium-based compounds. The contrast media are physically embedded into the biodegradable polymer matrix used. The disadvantage with this is that the physically incorporated radiopaque additives distinctly impair the processing and mechanical properties of the metal-free materials. In addition, the radiopaque additives described are nondegradable substances which all not are generally recognized as safe with respect to cytotoxicity. Hence, the metal powders added worsen the biocompatibility of the construction materials.

Occlusion instruments are medical devices used for closing defects of the septum in the heart or else for occluding an atrial auricle. These septal defects include particularly persistent Foramen ovale (PFO), atrial septal defects (ASDs) and ventricular septal defects (VSDs). These defects are closed using occluders which are constructed e.g. of two retention umbrellas and a waist in between. Previously used occluders are produced from a continuous wire braid or from a conical or spherical metal wire braid, the wires being made of the shape memory alloy Nitinol. Nitinol is a “shape memory alloy” based on nickel and titanium so comprising only moderate deformability, and can either itself or through appropriate corrosion products lead e.g. to allergic defense reactions on actual part of the body. By contrast, occluders based on biodegradable or bioresorbable polymers exhibit clearly higher deformabilities and improved biocompatibility. However, the main disadvantage is that the positioning of the occluders cannot be monitored using traditional diagnostics.

In the prior art, occlusion instruments, based on such biodegradable radiopaque polymers, are not known. Hence, there is a need for improved occlusion instruments. Further, in particular there is a need for occlusion instruments comprising advantageous bioresorbable and thermoplastically deformable polymers detectable by means of X-rays.

### SUMMARY OF THE INVENTION

An object of the invention is to overcome one or more of the above-mentioned disadvantages of conventional devices and/or provide occlusion instruments comprising biodegradable radiopaque polymers which preferably have the suitable strength and sufficient deformability, good bioresorbability and adequate visibility and hence particularly useful for producing metal-free occlusion instruments for closing defects of the septum in the heart, the positioning of which is monitorable using customary diagnostic methods.

According to the present invention, this object is achieved when the occlusion instrument comprises the features of claim 1.

Embodiments of the invention provides occlusion instruments comprising bioresorbable and thermoplastically deformable polymers, which polymers are advantageously biodegradable and/or bioresorbable polymer materials.

The polymers additionally have one or more of the following advantageous characteristics:
preoperatively simple to obtain and offering a range of possible variations,
processible by thermal processes or from solutions into fibers, film or shaped articles,
having requisite strength and sufficient deformability for collapsible occlusion instruments,
unexpected good bioresorbability,
sufficient visibility in X-ray light, and/or
particularly useful for producing metal-free occlusion instruments for closing defects of the septum in the heart, the positioning of which is monitorable using customary diagnostic methods.

Thus, an aspect of the invention relates to an occlusion instrument for closing defects, e.g. of the septum, in the heart, comprising bioresorbable, radiopaque and thermoplastically deformable polymers with or without shape memory characteristics, said polymers comprising radiopaque building groups in the repeat units of the polymer chains, and/or modified with bioresorbable, radiopaque nanoparticles.

In the context of the present invention, the term “polymers” can both relate to two or more polymer molecules of the same type or to two or more polymer molecules of different types.

Another aspect of the invention relates to a process for producing the occlusion instrument, wherein the bioresorbable, radiopaque and thermoplastically deformable polymers with or without shape memory characteristics, which on the one hand contain radiopaque triiodophenyl side groups conforming to the formula (I) in the repeat units of the polymer chains and/or on the other are modified with bioresorbable, radiopaque nanoparticles are solution or melt spun to produce threads which are subsequently processed by shaping processes into occlusion instruments.

A further aspect of the invention relates to monofils or multifilament yarns comprising bioresorbable, radiopaque and thermoplastically deformable polymers with or without shape memory characteristics, said polymers comprising radiopaque building groups in the repeat units of the polymer chains, and/or modified with bioresorbable, radiopaque nanoparticles, and said yarns being thermoplastic or non-meltable but soluble and processible from the melt or solution.

Yet an aspect of the invention relates to bioresorbable, radiopaque and thermoplastically deformable polymers with or without shape memory characteristics, said polymers comprising radiopaque building groups in the repeat units of the polymer chains, and/or modified with bioresorbable, radiopaque nanoparticles.

Yet further aspects of the invention relate to various uses of the polymers described herein, which polymers are thermoplastically deformable polymers with or without shape memory characteristics, said polymers comprising radiopaque building groups in the repeat units of the polymer chains, and/or modified with bioresorbable, radiopaque nanoparticles.

### SHORT DESCRIPTION OF THE DRAWINGS

In the following, some embodiments of the invention are described with reference to the drawings, wherein

FIG. 1 shows examples of commercial triiodophenyl derivatives;

FIG. 2 shows examples of commercial triiodophenyl derivatives;

FIG. 3 shows an example of synthesis of a diol containing triiodophenyl side groups;

FIG. 4 shows the diol PE-GA-D (TIPh) as an example of a building group with triiodophenyl side groups conforming to the hereinbelow indicated formula (I) (m=2, (L=H, A=C(CH₂—)₄);

FIG. 5 shows structures of biodegradable polyesters;

FIG. 6 shows structures of biodegradable polyanhydrides poly(amino acid)s or polyamides;

FIG. 7 shows structures of a radiopaque, biodegradable poly(lactic acid)-diol;

FIG. 8 shows possible syntheses for covalent, radiopaque, biodegradable polymer networks;

FIG. 9 Examples of known free-radically polymerizable triiodo monomers;

FIG. 10 Examples of a biodegradable polyorthoester and polyphosphazene;

FIG. 11 and FIG. 12 show braids constructed from radiopaque, biodegradable polymeric filaments using a braiding machine, and

FIG. 13 Illustrative forms of occlusion instruments, in the expanded state.

### DETAILED DESCRIPTION OF THE INVENTION

The skilled person will understand that obvious modifications of actual embodiments, of the invention here given, are possible without departing from the scope of the accompanying claims.

As above mentioned, the object achieved by the invention is solved in accordance with the embodiments of the invention by bioresorbable and thermoplastically deformable polymers with or without “shape memory” characteristics” such as

a) on the one hand, contain radiopaque groups in the repeat units of the polymer backbone, and/or
b) on the other, are modified with bioresorbable, radiopaque nanoparticles. Particularly described in what follows.

### a) Radiopaque Groups

In an embodiment of the invention, the bioresorbable, radiopaque and thermoplastically deformable polymers comprise triiodophenyl side groups as the radiopaque building groups, according to the formula (I):

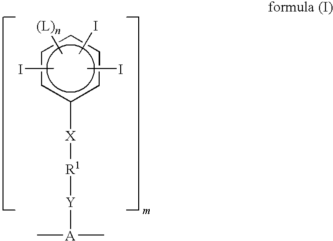

where
L represents H or a moiety comprising a functional group selected from the group consisting of a carboxylic acid, an amine, a phosphate, a phosphonic acid, a sulfate, a sulfonic acid, an oligo(ethylene oxide), an amide, protonated or deprotonated states thereof, and salts thereof,
n=0, 1, or 2,
Y and X are, independently, absent or represent bonding groups,
R¹ is absent or represents an at least 2-valent linear, branched or cycloorganic moiety comprising 1 to 15 carbon atoms,
A represents an m+2-valent linear, branched or cycloorganic moiety containing 1 to 30 carbon atoms, and
m=1, 2, 3, or 4, and
   the phenyl moiety is substituted with the iodine atoms in free o-, m- or p-positions.

It should be noted that A of formula (I) forms part of the backbone of the polymer. A preferred amide group is an acetylamino group. Useful bonding groups are e.g. a group containing an ether, a group containing a carboxylic ester, a group containing a carboxamide, or a urethane group.

In a particular embodiment
L represents H or a moiety comprising a functional group selected from the group consisting of a carboxylic acid, an amine, a phosphate, a sulfate, an oligo(ethylene oxide), an amide, protonated or deprotonated states thereof, and salts thereof,
n varies between 0, 1 or 2,
Y and X are, independently, absent or represent bonding groups, such as carboxylic ester or urethane groups,
R¹ is absent or represents an at least 2-valent linear, branched or cycloorganic moiety comprising from 1 to 10 carbon atoms,
A represents an m+2-valent linear, branched or cycloorganic moiety containing 1 to 20 carbon atoms,
m=1 or 2, and
   the phenyl moiety is substituted with the iodine atoms in free o-, m- or p-positions.

In another embodiment of the invention, the radiopaque, bioresorbable and thermoplastically deformable polymers are characterized in that they contain the following radiopaque building groups having triiodophenyl side groups conforming to the formula (I):

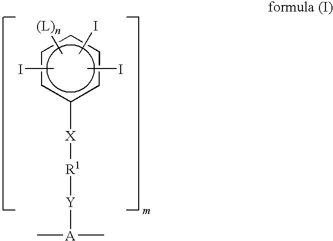

where
L represents H or a water-solubilizing carboxylate, ammonium, phosphate, phosphonate, sulfate or sulfonate group or an oligo(ethylene oxide) or acetylamino radical,
n can vary between 0, 1 or 2,
Y and X are absent or represent bonding groups, such as ether, carboxylic ester or carboxamide or urethane groups,
R¹ is absent or represents a 2-valent linear, branched or cycloorganic radical consisting of 1 to 15 carbon atoms,
A represents an m+2-valent linear, branched or cycloorganic radical containing 1 to 30 carbon atoms, and
m can vary between 1 and 4, and
   the phenyl radical can be substituted with the iodine atoms in free o-, m- or p-positions.

In a particular embodiment
L represents H or a water-solubilizing carboxylate, ammonium, phosphate, sulfate group or an oligoethylene oxide or acetylamino radical,
n varies between 0, 1 or 2,
Y and X are absent or represent bonding groups, such as carboxylic ester or urethane groups,
R¹ is absent or represents a 2-valent linear, branched or cycloorganic radical consisting of 1 to 10 carbon atoms,
A represents an m+2-valent linear, branched or cycloorganic radical containing 1 to 20 carbon atoms, and
m varies between 1 and 2, and
   the phenyl radical is substituted with the iodine atoms in free o-, m- or p-positions.

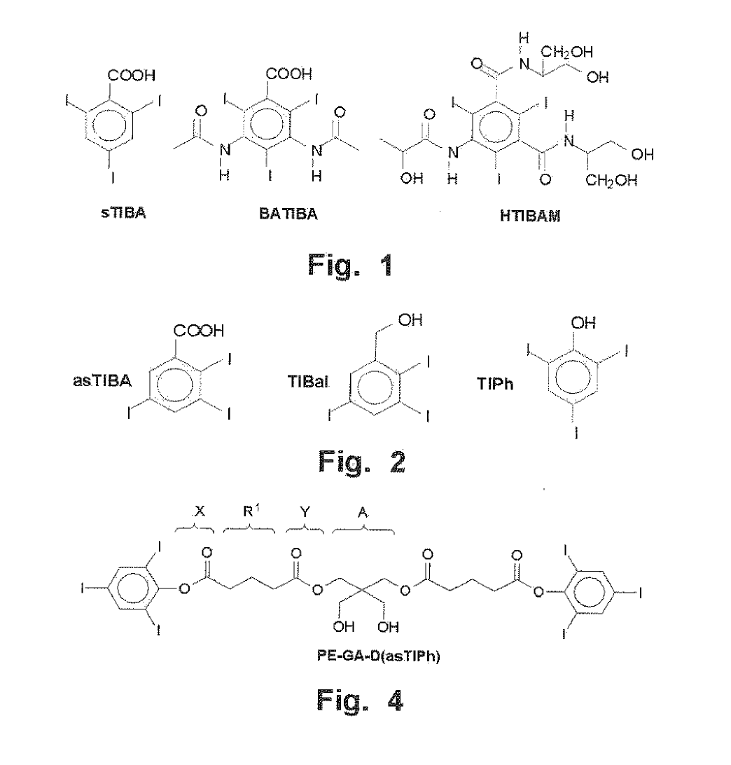

Furthermore, building groups having triiodophenyl side groups are particularly suitable when derived from commercial triiodophenyl derivatives e.g. 2,4,6-triiodobenzoic acid (sTIBA), 3,5-bis(acetamido)-2,4,6-triiodobenzoic acid (BATIBA, diatrizoic acid) or 5-(α-hydroxypropionylamino-2,4,6-triiodoisophthalic acid di(1,3-hydroxyisopropylamide) (HTIBAM, iopamidol), as illustrated in FIG. 1.

In addition, the following commercial triiodo compounds are possible for use as synthons: 2,3,5-triiodobenzoic acid (asTIBA), 2,3,5-triiodobenzyl alcohol (TIBal) or 2,4,6-triiodophenol (TIPh), see FIG. 2. These triiodophenyl derivatives can be reacted with suitable multifunctional organic compounds, if appropriate by utilizing protecting group technology, to produce suitable radiopaque building groups having triiodophenyl side groups conforming to the formula (I)

This method of preparation will now be elucidated using an example.

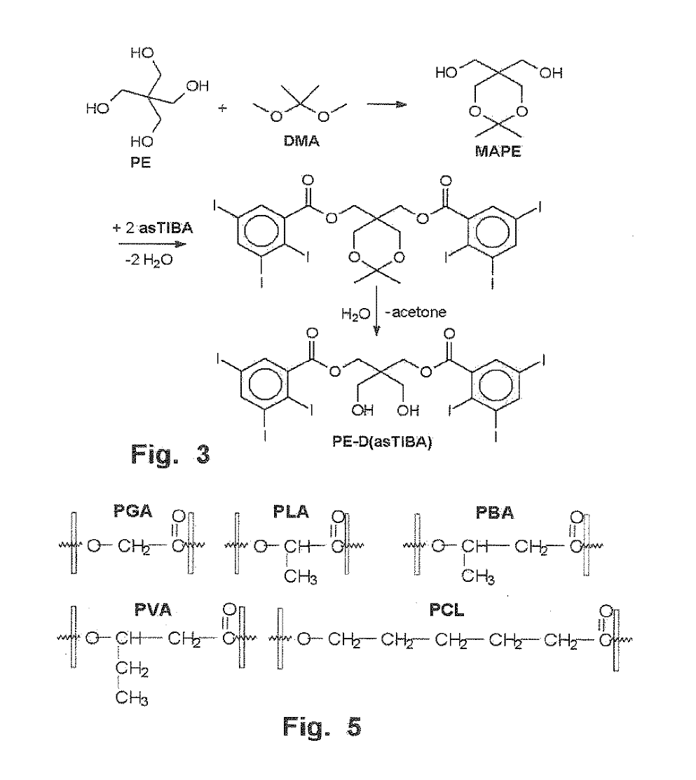

It comprises converting pentaerythritol (PE) in a 1^{st} stage by ketalization with dimethoxyacetone (DMA) into a monoacetone pentaerythritol (MAPE), then esterifying the two free OH groups in a 2^{nd} stage with 2,3,5-triiodobenzoic acid (asTIBA) and finally re-detaching the acetone protecting group in a 3^{rd} stage by acidic hydrolysis to form a diol containing two triiodophenyl side groups PE-D (asTIBA). This is illustrated in FIG. 3.

The synthesis may alternatively comprise esterifying (MADE) to incorporate a spacer initially with glutaric anhydride (GA), then incorporating the two triiodophenyl side groups by reaction with 2,4,6-triiodophenol (TIPh) and finally detaching the acetone protecting group in the last stage to again form the diol containing two triiodophenyl side groups PE-GA-D (TIPh), see FIG. 4.

There are embodiments of the invention where, as well as pentaerythritol, other multifunctional organic compounds having at least three identical or different functional groups can be used in a similar manner to produce the radiopaque building groups. The functional groups can for instance be hydroxyl, amino, thiol or carboxyl groups. Hydroxyl-containing groups having more than three hydroxyl groups per molecule appear to be particularly suitable. These compounds, as well as hydroxyl groups, may contain additional functional groups. Hydroxyl-containing compounds useful for preparing the radiopaque building groups of the invention as well as pentaerythritol comprise for example erythritol, xylitol, sorbitol, inositol, methylglucoside or quinic acid. In accordance with the identity and number of functional groups in these multifunctional compounds, radiopaque building groups according to the present invention can be synthesized by using suitable protecting group techniques as known in prior art.

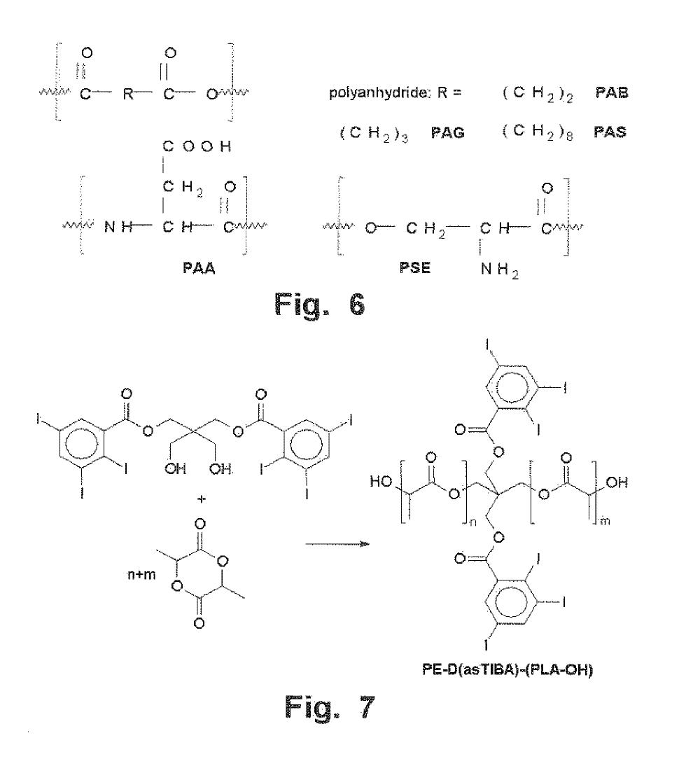

The radiopaque building groups of the formula (I) may be incorporated in bioresorbable polymers by copolymerization, co-condensation or polyaddition. The following known biodegradable synthetic classes of polymer (cf. J. M. Mayer, D. L. Kaplan, Trends Polym. Sci. 2 (1994) 227) can be used:
polyesters, such as poly(lactic acid) PLA, poly(glycolic acid) PGA, poly(3-hydroxybutyric acid) PBA, poly(4-hydroxyvaleric acid) PVA or poly(ε-caprolactone) PLC or corresponding copolymers, see FIG. 5;
polyanhydrides formed from dicarboxylic acids such as for example glutaric PAG, succinic PAB or sebacic acid PAS see FIG. 6; or
poly(amino acid)s or polyamides such as for example poly(serine ester) PSE or poly(aspartic acid) PAA see FIG. 6.

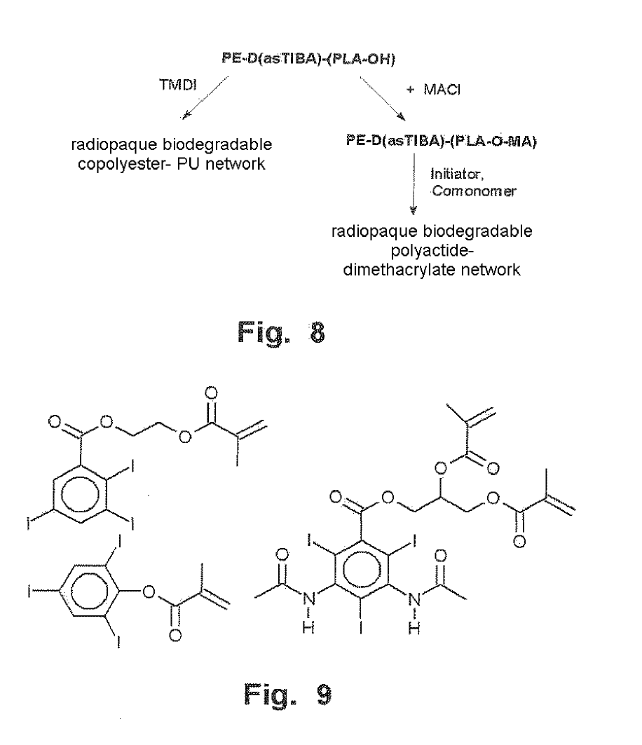

The radiopaque building groups of the formula (I) such as for example the diol PE-D (asTIBA) may be used as a starter alcohol for the ring-opening polymerization of lactide for example. The resulting OH-terminated biodegradable, radiopaque polymers PE-D (asTIBA)-(PLA-OH), see FIG. 7, can then be crosslinked with commercial diisocyanates, for example trimethylhexamethylene diisocyanate (TMDI), to form a biodegradable polyurethane network, as depicted in FIG. 8.

The OH-terminated, biodegradable, radiopaque polymers PE-D (asTIBA)-(PLA-OH) may further be converted by reaction with, e.g. methacryloyl chloride (MACl) into free-radically polymerizable, biodegradable, radiopaque telechels. These telechels may then be copolymerized in the presence of a free-radical initiator and if appropriate of further free-radically polymerizable co-monomers, in which case a biodegradable, radiopaque, covalent polymer network is formed. Advantageous co-monomers are known radiopaque mono- or dimethacrylates which are readily commercially available and known to be biocompatible, see FIG. 9 and cf. N. Moszner, U. Salz, A. M. Klester, V. Rheinberger, Angew. Macromol. Chem. 224 (1995) 115, K. W. M. Davy, M. R. Anseau, M. Odlyha, G. M. Foster, Polym. Intern. 43 (1997) 143. An advantage is that co-monomers of known reactivity, and radiopaque capacity, can be used. In this manner the manufacturing process thereof may be simplified.

Cross-linking may also take place after any thread formation of the components or after the production of shaped devices such as occlusion instruments for example. A radiation-induced cross-linking using high-energy radiation such as γ-radiation for example is particularly useful. A further improvement in the mechanical properties, i.e. in breaking strength or in modulus of elasticity, may be achieved by properly selection of radiation conditions, e.g. expected radiation dose.

It is of advantage for the bioresorbability of the radiopaque, thermoplastically deformable polymers that the radiopaque building groups having triiodophenyl side groups conforming to the formula (I), which are released due to biodegradation, have sufficient solubility in water. One advantage of water-soluble triiodophenyl side groups is that they lead to particularly readily bioresorbable polymers. This can be achieved through the incorporation of water-solubilizing carboxylate, ammonium, phosphate, phosphonate, sulfate or sulfonate groups or of oligo(ethylene oxide) or acetylamino moieties.

Additives may be added to the radiopaque, thermoplastically deformable polymer(s) before or after shaping to modify and adjust the mechanical, thermal and/or specific properties. When so, additives like plasticizers, polymeric or low molecular weight organic fillers, dyes, biodegradation-influencing substances, or organic or inorganic compounds, which additionally improve the radiopaque property, can be used.

b) Modification with Radiopaque Nanoparticles

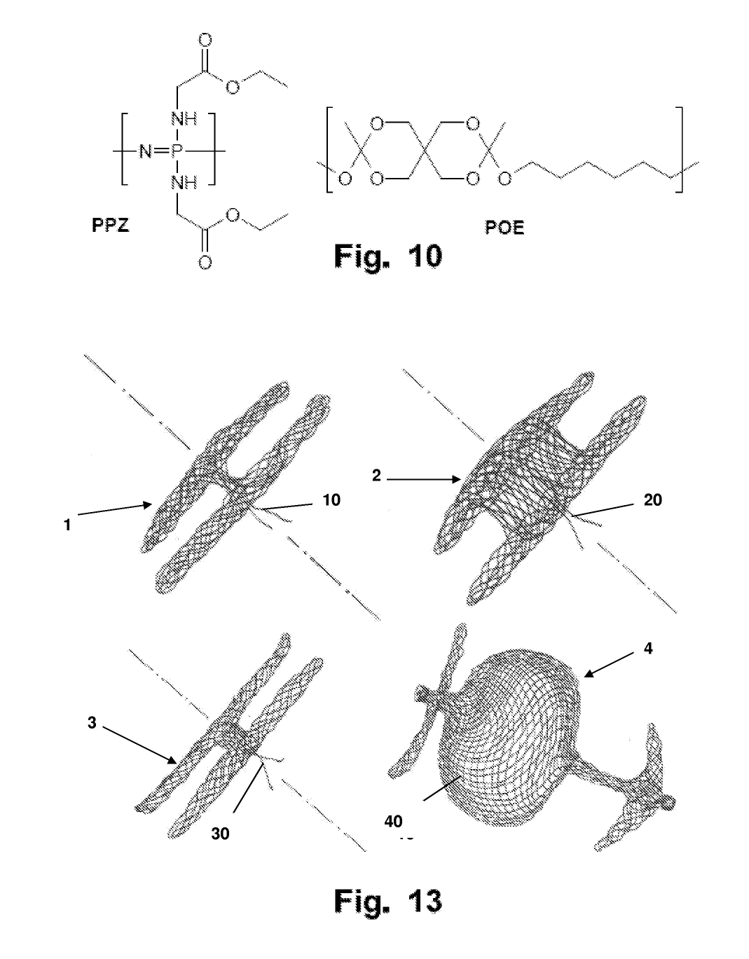

The modification according to embodiments of the present invention of the bioresorbable and thermoplastically deformable polymers with or without “shape memory characteristics” may be effected using bioresorbable, radiopaque nanoparticles. These nanoparticles may be produced by nanoencapsulation of bioresorbable, radiopaque compounds, i.e. so affecting the electron density. The micro- or nanoencapsulation of finely disperse liquid or solid components by envelopment with film-forming polymers is a known technology used for example for protecting less stable components against ambient influences, to reduce the odor of malodorous components or to manufacture medicaments having a controlled drug release profile, cf. C. A. Finch, in: Ullmann's Encyclopedia of Industrial Chemistry, 4 Ed., Vol. A16, VCH Verlagsgesellschaft, Weinheim etc. 1993, 575; C. Thies, in: Encyclopedia of Polymer Science and Engineering, Vol. 9. John Wiley & Sons, New York etc. 1988, 724. However, this is hitherto not employed for implants, such as occlusion devices. Depending on the method of making employed, the nanocapsules required for particular embodiments of the present invention's modification of the bioresorbable and thermoplastically deformable polymers can be produced in a size of about to 1000 nm. The individual methods of making nanocapsules differ according to whether monomers or polymers are used as starting materials to form the wall, and whether the wall-formers are present in one of the phases (core phase or continuous phase) or in both. It is usually not necessary for perfectly spherical capsules to be formed. The particularly small nanocapsules may be produced via so-called miniemulsions, cf. N. Bechthold, F. Tiarks, M. Willert, K. Landfester, M. Antonietti, Marcomol. Symp. 2000, 151, 549. For the present invention's bioresorbable, radiopaque nanoparticles, it is advantageous to use a water-soluble or biodegradable polymer as enveloping material. The advantage of using water-soluble or biodegradable enveloping polymers is that they lead to particularly readily bioresorbable nanoparticles. Useful water-soluble polymers include for example commercially available starch or cellulose derivatives, for example sodium alginate or carboxymethylcellulose, and also pullulan, polyvinyl alcohol or gelatin. Particularly useful non-water-soluble, biodegradable polymers are according to embodiments of the present invention the abovementioned polyesters based on α-hydroxy carboxylic acids, such as lactic acid or glycolic acid, and also their copolymers, polyanhydrides and poly(α-amino acid)s. It is also possible to use polyorthoesters (POEs) or polyphosphazenes (PPZs) cf. FIG. 10.

The core material for the bioresorbable, radiopaque nanoparticles may in embodiments be suitable derivatives of commercial triiodophenyl compounds, for example 2, 4,6-triiodobenzoic acid (sTIBA), 3,5-bis(acetamido)-2,4,6-triiodobenzoic acid (BATIBA, diatrizoic acid), 5-(α-hydroxypropionylamino-2,4,6-triiodoisophthalic acid di(1,3-hydroxyisopropylamide) (HTIBAM, iopamidol) (FIG. 1), 2,3,5-triiodobenzoic acid (asTIBA), 2,3,5-triiodobenzyl alcohol (TIBal) or 2,4,6-triiodophenol(TIPh) (FIG. 2).

The specific selection of certain polymers makes it possible to adjust the rate of biodegradability or bioresorption, the swellability, the stability and mechanical properties of the polymeric nanocapsules to specific values.

Melt or solution spinning of the bioresorbable and thermoplastically deformable polymers with or without shape memory characteristics of the present invention, which on the one hand contain radiopaque triiodophenyl side groups conforming to the above formula (I) in the repeat units of the polymer chains and/or on the other are modified with bioresorbable, radiopaque nanoparticles, may be used to produce threads. These threads may consist not only of a single thread, a so-called monofil, but also of a plurality of individual filaments, which then form a so-called multifilament (also known as multifilament yarn), monofils usually being thicker than individual filaments of the multifilament yarns. Threads formed from the melt are cooled down after spinning and the heat transfer which takes place in the process is improved by cooled media. The strength of the filaments obtained is if necessary enhanced by drawing, further processes for enhancing the strength are thermal treatment, crosslinking or else combinations thereof. Drawing can also be augmented by heating the threads, in which case the heating may be effected by air or other gases, for example nitrogen, liquids or radiation (microwaves, IR radiation).

The coherency of the assembly of individual filaments which forms the multifilament yarn may be improved by application of twists and/or by entangling. As well as these techniques for improving coherency, the use of adhesive substances is also possible. It is then possible to use the threads of bioresorbable, radiopaque polymers to produce corresponding texture, or else other suitable form, for occlusion instruments.

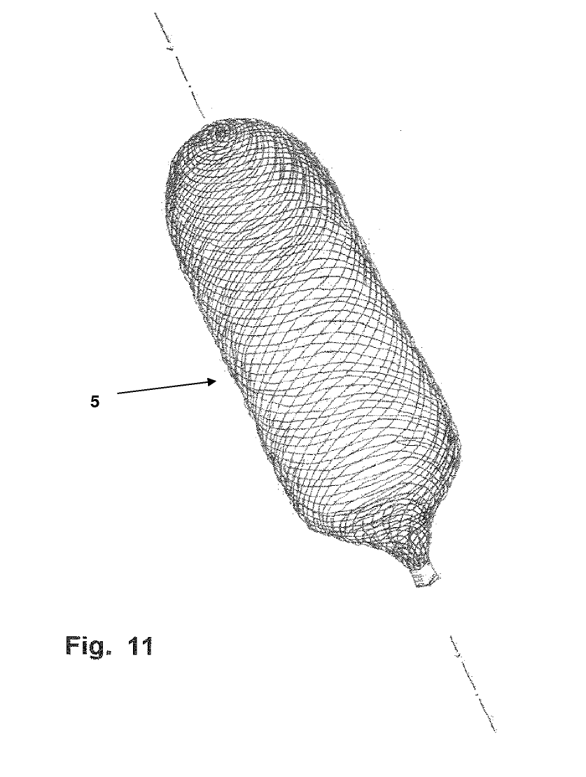

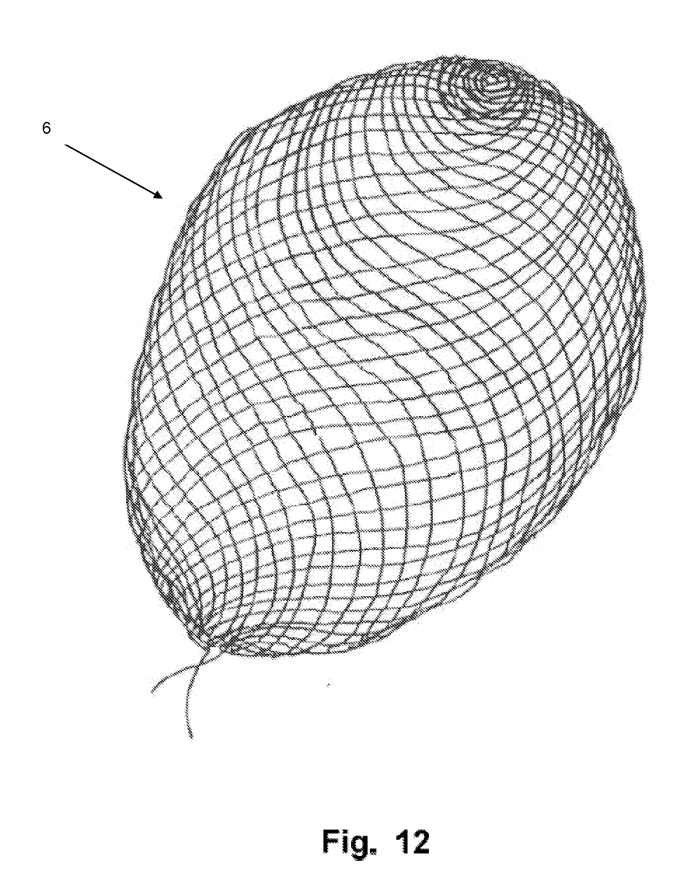

Initially flexible continuous braids or flexible funnel- or sphere/pear/drop-shaped braid 5, 6 are formed by means of a braiding machine, see FIG. 11 and FIG. 12. Yarns 10, 20, 30, 40, or more particularly, multi- or monofilaments can be used for braid making. The funnel- or sphere/pear/drop-shaped braids 5, 6 can be bundled and encased at one end using known braiding technology, see DE 10338702; DE 102006013770. When continuous braids are produced, both ends of the braid are bundled and encased. The braided fabric is then brought into the desired shape by a heat-treating step, the shape which is conferred being dictated by the engineered design of the device. Particularly inductive or thermal heat-treating methods can here be used. Heat treatment time and temperature is chosen such that the braided fabric retains its conferred shape. After heat treatment, the shape-conferring device is removed. The braid retains its conferred shape. The braided fabric thus treated corresponds to the previously fixed (expanded) shape of the medical occlusion instrument, which can be implanted in its collapsed state by means of a catheter system. Some exemplary forms of such occlusion instruments 1, 2, 3, 4 comprising yarns 10, 20, 30, 40 respectively are depicted in FIG. 13.

In embodiments of the invention, the occlusion instrument 1, 2, 3, 4 comprises the bioresorbable, radiopaque and thermoplastically deformable polymers in an amount in the range of 5-100% by weight of the occlusion instrument, such as in the range of 25-100%, preferably in the range of 50-100% by weight of the occlusion instrument, such as in the range of 75-100%, and even more preferably in the range of 80-100% by weight of the occlusion instrument, such as in the range of 95-100% by weight of the occlusion instrument.

In an embodiment of the invention, the occlusion instrument 1, 2, 3, 4 essentially consists of the bioresorbable, radiopaque and thermoplastically deformable polymers.

In the context of the present invention, the term “essentially consists of” means the occlusion instrument 1, 2, 3, 4 mainly consists of the polymers as described herein, and that the occlusion instrument 1, 2, 3, 4 may contain other components, such as e.g. fabric, additives, and/or pharmaceuticals.

It is preferred that the occlusion instrument of the present invention is radiopaque.

In some embodiments of the invention, the occlusion instrument comprises less than 5% metal and metal alloy by weight of the occlusion instrument, and preferably substantially no metal and substantially no metal alloy.

In a preferred embodiment of the invention, the occlusion instrument comprises one more processed wires or strands comprising bioresorbable, radiopaque and thermoplastically deformable polymers. Such one or more wires or strands may be a yarn 10, 20, 30, 40 as described herein.

“Processing” in the context of the processed wires or yarns means that the wires or yarns have been processed into the final design of the occlusion instrument, which could be a braid design made of the wires or strands.

In embodiments, the one or more processed wires or strands may comprise the bioresorbable, radiopaque and thermoplastically deformable polymers in an amount in the range of 5-100% by weight of the one or more processed wires, such as in the range of 25-100%, preferably in the range of 50-100% by weight of the one or more processed wires, such as in the range of 75-100%, and even more preferably in the range of 80-100% by weight of the one or more processed wires, such as in the range of 95-100% by weight of the one or more processed wires.

In an embodiment of the invention, the occlusion instrument furthermore comprises one or more pharmaceuticals. An example of a useful pharmaceutical is an endothelialisation agent providing a particular dense and/or quick occlusion of occlusion devices upon implantation.

The occlusion instrument may furthermore comprise a fabric, e.g. comprising polymeric fibers such as cellulose fibers or polyester fibers. The fabric is preferably bioresorbable and/or biodegradable. The fabric may improve endothelialisation and thus a quick occlusion of occlusion devices upon implantation.

Exemplary embodiments of the invention are furthermore described below:

### Exemplary Embodiment 1

A bioresorbable and thermoplastically deformable polymers with or without shape memory characteristics, comprising radiopaque building groups in the repeat units of the polymer chains, and/or modified with bioresorbable, radiopaque nanoparticles.

### Exemplary Embodiment 2

Polymers according to Exemplary embodiment 1, wherein the aforementioned, bioresorbable and thermoplastically deformable polymers comprise triiodophenyl side groups as the aforementioned radiopaque building groups, conforming to the formula (I):

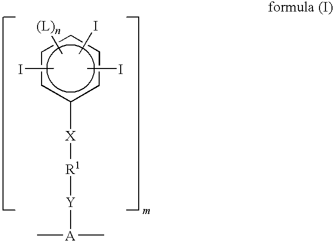

where
L represents H or a water-solubilizing carboxylate, ammonium, phosphate, phosphonate, sulfate or sulfonate group or an oligo(ethylene oxide) or acetylamino radical,
n can vary between 0, 1 or 2,
Y and X are absent or represent bonding groups, such as ether, carboxylic ester or carboxamide or urethane groups,
R¹ is absent or represents a 2-valent linear, branched or cycloorganic radical consisting of 1 to 15 carbon atoms,
A represents an m+2-valent linear, branched or cycloorganic radical containing 1 to 30 carbon atoms, and
m can vary between 1 and 4, and
the phenyl radical is substituted with the iodine atoms in free o-, m- or p-positions.

### Exemplary Embodiment 3

Polymers according to Exemplary embodiment 1, wherein the aforementioned radiopaque, bioresorbable and thermoplastically deformable polymers contain triiodophenyl side groups as the aforementioned radiopaque building groups, conforming to the formula (I):

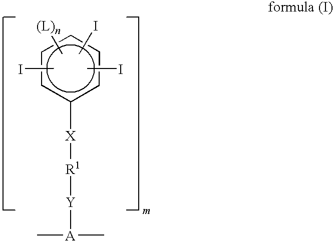

where
L represents H or a water-solubilizing carboxylate, ammonium, phosphate, sulfate group or an oligoethylene oxide or acetylamino radical,
n varies between 0, 1 or 2,
Y and X are absent or represent bonding groups, such as carboxylic ester or urethane groups,
R¹ is absent or represents a 2-valent linear, branched or cycloorganic radical consisting of 1 to 10 carbon atoms,
A represents an m+2-valent linear, branched or cycloorganic radical containing 1 to 20 carbon atoms, and
m varies between 1 and 2, and
the phenyl radical is substituted with the iodine atoms in free o-, m- or p-positions.

Exemplary Embodiment 4

Bioresorbable and thermoplastically deformable polymers according to Exemplary embodiment 1, wherein the radiopaque nanoparticles represent nanocapsules constructed of a core of radiopaque triiodophenyl compounds and an envelope of bioresorbable polymers.

Exemplary Embodiment 5

Bioresorbable and thermoplastically deformable polymers according to any of the preceding Exemplary embodiments, wherein the radiopaque building groups are incorporated in the bioresorbable polymers by: copolymerization, co-condensation or poly-addition.

Exemplary Embodiment 6

Bioresorbable and thermoplastically deformable polymers according to Exemplary embodiment 5, wherein the bioresorbable polymers comprise polyesters, polyanhydrides, polycarbonates, polyamides or polyamino acids.

Exemplary Embodiment 7

Bioresorbable and thermoplastically deformable polymers according to Exemplary embodiment 6, wherein the polyesters comprise poly(α-hydroxy carboxylic acid)s or copolymers thereof.

### Exemplary Embodiment 8

Bioresorbable and thermoplastically deformable polymers according to any of the preceding Exemplary embodiments, wherein the uncross-linked radiopaque polymers formed after incorporation of the radiopaque building groups in the bioresorbable polymers are cross-linked by diisocyanates.

### Exemplary Embodiment 9

Bioresorbable and thermoplastically deformable polymers according to any of the preceding Exemplary embodiments, wherein the uncross-linked radiopaque polymers formed after incorporation of the radiopaque building groups in the bioresorbable polymers are provided with free-radically polymerizable groups by end group modification and the telechels formed are subsequently copolymerized in the presence of a free-radical initiator and if appropriate one or more free-radically polymerizable co-monomers to form a biodegradable, radiopaque polymer network.

### Exemplary embodiment 10

Bioresorbable and thermoplastically deformable polymers according to any of the preceding Exemplary embodiments, wherein the bioresorbable and thermoplastically deformable polymers are admixed, before or during shaping, with additives which effect an adjustment and adaptation of the mechanical thermal properties and/or specific application properties of the polymers.

### Exemplary embodiment 11

Process for producing bioresorbable and thermoplastically deformable polymers with or without shape memory characteristics according to any of Exemplary embodiments 1-13 (1-10, 12, 13), comprising introducing radiopaque building groups into the repeat units of the polymer chain and/or modifying the polymers with bioresorbable, radiopaque nanoparticles.

### Exemplary embodiment 12

Process, in accordance with Exemplary embodiment 11, for producing bioresorbable and thermoplastically deformable polymers according to Exemplary embodiment 4, wherein the radiopaque nanoparticles represent nanocapsules constructed of a core of radiopaque triiodophenyl compounds and an envelope of bioresorbable polymers, the process comprising using non-water-soluble, biodegradable polyesters based on α-hydroxy carboxylic acids, their copolymers, polyanhydrides, poly(α-amino acid)s, polyorthoesters and/or comprising using polyphosphazenes as envelope materials for producing the radiopaque nanocapsules.

### Exemplary embodiment 13

Process, according to Exemplary embodiment 11, for producing bioresorbable and thermoplastically deformable polymers according to Exemplary embodiments 1, 2 or 3, comprising using multifunctional organic compounds for producing the radiopaque building groups, the multifunctional organic compounds having at least three identical or different functional groups.

### Exemplary embodiment 14

Process according to Exemplary embodiment 13, wherein the radiopaque building groups are produced using hydroxyl-containing compounds having more than three hydroxyl groups per molecule and wherein these hydroxyl-containing compounds may contain further functional groups.

### Exemplary Embodiment 15

Process according to Exemplary embodiment 11, comprising incorporating the radiopaque building groups in the bioresorbable polymers by: copolymerization, cocondensation or polyaddition.

### Exemplary Embodiment 16

Process according to any one of Exemplary embodiments 11-15, wherein uncross-linked radiopaque polymers formed after incorporation of the radiopaque building groups in the bioresorbable polymers are cross-linked by diisocyanates.

### Exemplary Embodiment 17

Process according to any one of Exemplary embodiments 11-16, wherein the uncross-linked radiopaque polymers formed after incorporation of the radiopaque building groups in the bioresorbable polymers are provided with free-radically polymerizable groups by end group modification and the telechels formed are subsequently copolymerized in the presence of a free-radical initiator and if appropriate one or more free-radically polymerizable co-monomers to form a biodegradable, radiopaque polymer network.

### Exemplary Embodiment 18

Process according to any of Exemplary embodiments 11-17, wherein the bioresorbable and thermoplastically deformable polymers are admixed, before or during shaping, with additives which effect an adjustment and adaptation of the mechanical thermal properties and/or specific application properties of the polymers.

### Exemplary Embodiment 19

Process according to Exemplary embodiment 11, comprising converting pentaerythritol (PE) in a 1st stage by ketalization with dimethoxyacetone (DMA) into a monoacetone pentaerythritol (MAPE), then esterifying the two free OH groups in a 2nd stage with 2,3,5-triiodobenzoic acid (asTIBA) and redetaching the acetone protecting group in a 3rd stage by acidic hydrolysis to form a diol containing two triiodophenyl side groups PE-D (asTIBA) as illustrated in FIG. 3.

### Exemplary Embodiment 20

Process according to Exemplary embodiment 19, alternatively comprising esterifying monoacetone pentaerythritol (MAPE) to incorporate a spacer with glutaric anhydride (GA), then incorporating the two triiodophenyl side groups by reaction with 2,4,6-triiodophenol (TIPh) and at last detaching the acetone protecting group in the last stage to again form the diol containing two triiodophenyl side groups PE-GA-D (TIPh).

### Exemplary Embodiment 21

Monofils or multifilament yarns comprising bioresorbable and thermoplastically deformable polymers produced according to Exemplary embodiments 1-4 and being thermoplastic or non-meltable but soluble and processible from the melt or solution.

### Exemplary Embodiment 22

Process for producing monofils or multifilament yarns according to Exemplary embodiment 21, wherein the monofils or multifilament yarns are produced from bioresorbable and thermoplastically deformable polymers according to Exemplary embodiment 1-4 by using an extrusion spinning apparatus.

### Exemplary Embodiment 23

Process for producing monofils or multifilament yarns according to Exemplary embodiment 21, wherein the monofils or multifilament yarns are produced from bioresorbable and thermoplastically deformable polymers according to Exemplary embodiments 1-4 by using a plunger-type spinning apparatus.

### Exemplary Embodiment 24

Process for producing monofils or multifilament yarns according to Exemplary embodiment 21, wherein the monofils or multifilament yarns are produced from bioresorbable and thermoplastically deformable polymers according to Exemplary embodiment 1-4 by using a spinning apparatus, in particular by solution-spinning apparatuses.

### Exemplary Embodiment 25

Process for producing monofils or multifilament yarns according to Exemplary embodiment 21, wherein the monofils or multifilament yarns produced from bioresorbable and thermoplastically deformable polymers according to Exemplary embodiment 1-4 are consolidated by a drawing operation carried out after the spinning operation.

### Exemplary Embodiment 26

Process for producing monofils or multifilament yarns according to Exemplary embodiment 25, wherein the spinning and drawing operations are simultaneously combined as subsidiary operations in one process step or are carried out as sequentially independent operations at different places and/or times.

### Exemplary Embodiment 27

Process for producing monofils or multifilament yarns according to Exemplary embodiment 21, wherein the monofils or multifilament yarns are of the bioresorbable and thermoplastically deformable polymers according to Exemplary embodiment 1 to 4, comprising consolidating the monofils or multifilament yarns by heat treating in which heat is transferred through hot gases or mixtures of gases, hot liquids or by radiation.

### Exemplary Embodiment 28

Process for producing monofils or multifilament yarns according to Exemplary embodiment 24, wherein the monofils or multifilament yarns are consolidated by cross-linking initiated by radiation particularly by γ- or UV-radiation.

### Exemplary Embodiment 29

Process for producing multifilament yarns according to Exemplary embodiment 22, wherein the coherency of the filament assembly of the multifilament yarn is stabilized and improved by applying twists.

### Exemplary Embodiment 30

Process for producing multifilament yarns according to Exemplary embodiment 22, wherein the coherency of the filament assembly of the multifilament yarn is stabilized and improved by entangling and forming yarn entanglements.

### Exemplary Embodiment 31

Process for producing multifilament yarns according to Exemplary embodiment 22, wherein the coherency of the filament assembly of the multifilament yarn is stabilized and improved by application of adhesive substances, such as size, spin finish or some other finish.

### Exemplary Embodiment 32

Occlusion instrument for closing defects of the septum in the heart, comprising a bioresorbable and thermoplastically deformable polymer according to any one of Exemplary embodiment 1-10.

### Exemplary Embodiment 33

Occlusion instrument according to Exemplary embodiment 32, produced by a process according to any of Exemplary embodiments 22-31.

### Exemplary Embodiment 34

Process for producing occlusion instruments according to Exemplary embodiment 32, wherein the bioresorbable and thermoplastically deformable polymers with or without shape memory characteristics, which on the one hand contain radiopaque triiodophenyl side groups conforming to the formula (I) in the repeat units of the polymer chains and/or on the other are modified with bioresorbable, radiopaque nanoparticles are solution or melt spun to produce threads which are subsequently processed by shaping processes into occlusion instruments.

### Exemplary Embodiment 35

Use of bioresorbable and thermoplastically deformable polymers according to Exemplary embodiments 1-4 for producing collapsible occlusion instruments, surgical articles or implants.

### Exemplary Embodiment 36

Use of bioresorbable and thermoplastically deformable polymers according to Exemplary embodiments 1-4 for producing collapsible occlusion instruments used for closing defects of the septum in the heart, the positioning of which being monitorable using radiodiagnostic methods.

### Exemplary Embodiment 37

Use of bioresorbable and thermoplastically deformable polymers according to Exemplary embodiment 4 for producing occlusion instruments, surgical articles or implants, wherein the production of the radiopaque nanocapsules utilizes water-soluble bioresorbable polysaccharides, polysaccharide derivatives, proteins or polyvinyl alcohols as enveloping materials.

### EXAMPLES

The invention will now be further clarified and described with references to specific examples.

### Example 1

### Synthesis of PE-D (asTIBA)

50 g (0.1 mol) of triiodobenzoic acid, 8.81 g (0.05 mol) of monoacetone pentaerythritol and 12.22 g (0.1 mol) of dimethylaminopyridine (DMAP) were dissolved in dry methylene chloride. 20.63 g (0.1 mol) of dicyclohexylcarbodiimide (DCC) were added spatula-wise and stirred with ice cooling. A white precipitate of dicyclohexylurea (DCH) precipitated within a few minutes. Stirring was continued at room temperature for a further 15 hours and the solid material was filtered off. It was repeatedly washed with THF. The combined solutions were concentrated to obtain a viscous brown oil which was admixed with ethyl acetate to produce a white precipitate which was filtered off and subsequently further purified by column chromatography (silica gel 60, mobile phase: 1:1 ethyl acetate/heptane). Yield: 65%. Melting point: 142° C. Iodide determination by elemental analysis: I: 66.51% (calculated: 66.80%).

To detach the isopropylidene protecting group, the intermediate obtained was admixed with an excess of 1N HCl in THF and stirred at room temperature for 24 hours. To workup, the THF was distilled off and the product taken up in methylene chloride. The organic phase was extracted twice with 100 ml each time of saturated NaHCO3 solution and concentrated salt water and then dried over sodium sulfate. After filtration and concentrating of the solution, the product mixture was worked up by column chromatography (silica gel 60, mobile phase: 1:1 ethyl acetate/heptane) to obtain the product in 60% yield in the form of a white power (melting point: 146° C.). Iodide determination by elemental analysis: I: 70.99% (calculated: 69.24%).

¹H NMR (ppm, THF-D₈): 3.57 and 3.59 (d, 4H, CH₂), 3.85-3.87 (t, 2H, OH), 4.31 (s, 4H, CH₂), 7.65 (s, 2H, CH) and 8.26 (s, 2H CH).

IR (cm⁻¹, KBr): 3296 (OH stretching vibration); 3067 (C—H stretching vibration); 1733 and 1709 (C═O stretching vibration); 1178 (C—O stretching vibration of COOH group); 1056 (C—O stretching vibration of CH₂OH group).

### Example 2

### Synthesis of PE-D (asTIBA)-(PLA-O-PAS)

A mixture of 1.0 g (0.9 mmol) of PE-D (asTIBA), 3.93 g (27.3 mmol) of lactide and 11 mg of tin(II) 2-ethylhexanoate were stirred for 3 h at 150° C. in the absence of moisture. After cooling the mixture to room temperature, 50 ml of dried methylene chloride were added followed by stirring until completely dissolved. Then, 5 ml of anhydrous triethylamine were added followed by the dropwise addition of 0.22 g (0.9 mmol) of sebacic dichloride with ice cooling, moisture exclusion and stirring. The reaction mixture was warmed to room temperature with stirring and stirred for a further 15 h. After addition of 50 ml of methylene chloride, the precipitated triethylammonium hydrochloride was filtered off and the residue washed with little methylene chloride. Then, the reaction mixture was extracted twice in succession with 120 ml each time of 1N hydrochloric acid, twice with 120 ml each time of saturated NaHCO3 solution and three times with each time 200 ml of water. The extract was dried over anhydrous sodium sulfate, filtered and concentrated under reduced pressure to leave a yellow solid in a yield of 80%. Molar mass determination by GPC gave: Mn: 29.600 g/mol; Mw: 43.500 g/mol; D: 1.47 (UV detection). The polymer obtained can be solution or melt spun to form threads. Iodide determination by elemental analysis revealed: I: 13.77% (calculated: 13.60%). This iodine content coupled with a specimen thickness of about 0.77 mm leads to a radiocapacity of 95% aluminium (Al). Hence the radiocapacity of the polymers is in the region of nitinol occluders, which have different radiocapacities over the entire occluder in that the radiocapacity is about 60% Al, in the interior of the occluder, about 80% Al in the middle and about 168% Al in the edge region.

### Example 3

### Production Variants of Mono- or Multifilaments

a) The polymer or polymer compound was melted with an extruder, the temperature of the melt being 5 to 15° C. above the melting range, i.e. determined by DSC or hot-stage microscopy. The melt was gear pumped through the drill-hole of a spinneret die for monofilaments, cooled in a water bath and withdrawn at a speed of 10 to 75 m/min. This monofil was drawn up to 20 fold in the water bath at temperatures of 30 to 80° C. to obtain a drawn monofilament having a diameter of about 0.05 mm and strengths of more than 40 cN/tex.
b) The polymer or polymer compound was melted in an extruder wherein the temperature of the melt being 5 to 15° C. above the melting range determined by DSC or hot-stage microscopy. The melt was gear pumped through the drill-holes of a spinneret die for multifilament yarns, cooled in a water bath and withdrawn at a speed of 10 to 75 m/min. This multifilament yarn was drawn up to 10 fold in the water bath at temperatures of 30 to 80° C. to obtain a drawn multifilament yarn having diameters of 25 μm for the individual filaments.
c) The polymer or polymer compound was melted with an extruder, the temperature of the melt being 5 to 15° C. above the melting range determined by DSC or hot-stage microscopy. The melt was gear pumped through the drill-holes of a spinneret die for multifilament yarns, cooled in flowing gases and withdrawn at a speed of 20 to 750 m/min. This multifilament yarn was drawn up to 8 fold in the water bath at temperatures of 30 to 80° C. to obtain a drawn multifilament yarn having diameters of 15 μm for the individual filaments and strengths of more than 40 cN/tex (>290 MPa).
d) The polymer or polymer compound was dissolved in methyl ether ketone to prepare an 8% solution. The solution was gear pumped through the drill-holes of a spinneret die for multifilament yarns, coagulated in a coagulation bath and withdrawn at a speed of 10 to 50 m/min. This multifilament yarn was drawn up to 10 fold in a water bath at temperatures of 30 to 80° C. to obtain a drawn multifilament yarn having diameters of 15 μm for the individual filaments and strengths>22 cN/tex.
e) The polymer or polymer compound was melted with a plunger-type spinning apparatus, the temperature of the melt being 5 to 15° C. above the melting range determined by DSC or hot-stage microscopy. The melt was plunger pressed through the drill-hole of a spinneret die for monofilaments, cooled down in the water bath and withdrawn at a speed of 5 to 75 m/min. This monofil was drawn up to 20 fold in a water bath at temperatures of to 80° C. to obtain a drawn monofilament having a diameter of 0.04 mm and strengths of more than 35 cN/tex.
f) The polymer or polymer compound was dissolved in acetone to prepare a 6% solution. The solution was plunger pressed through the drill-holes of a spinneret die for multifilament yarns by means of a plunger-type spinning apparatus, coagulated in a coagulation bath and withdrawn at a speed of 10 to 50 m/min. This multifilament yarn was drawn up to 10 fold in a water bath at temperatures of 30 to 80° C. to obtain a drawn multifilament yarns having diameters of 12 μm for the individual filaments and strengths of more than 25 cN/tex.
g) The polymer or polymer compound was used with the same substances as described in Example 3f) to prepare a 4.37% solution. The solution was gear pumped through the drill-holes of a spinneret die for multifilament yarns, coagulated in a coagulation bath and withdrawn at a speed of 5 to 105 m/min. This multifilament yarn was drawn up to 10 fold in a water bath at temperatures of 30 to 80° C. to obtain a drawn multifilament yarn having diameters of 15 μm for the individual filaments and strengths of more than 28 cN/tex.

The present invention has been described above with reference to specific embodiments. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims as well as equivalents thereof.

1. An occlusion instrument devised for closing defects of the septum in the heart, comprising a yarn of bioresorbable, radiopaque and thermoplastically deformable polymers with or without shape memory characteristics, said polymers comprising radiopaque building groups in the repeat units of the polymer chains,
and wherein said occlusion instrument furthermore comprises a bioresorbable fabric.

2. The occlusion instrument according to claim 1, wherein the occlusion instrument comprises the bioresorbable, radiopaque and thermoplastically deformable polymers in an amount in the range of 5-100% by weight of the occlusion instrument.

3. The occlusion instrument according to claim 1, wherein the occlusion instrument essentially consists of the bioresorbable, radiopaque and thermoplastically deformable polymers.

4. The occlusion instrument according to claim 1, wherein the occlusion instrument comprises one more processed wires, such as one or more processed yarns, comprising bioresorbable, radiopaque and thermoplastically deformable polymers.

5. The occlusion instrument according to claim 4, wherein the one or more processed wires comprise the bioresorbable, radiopaque and thermoplastically deformable polymers in an amount in the range of 5-100% by weight of the one or more processed wires.

6. The occlusion instrument according to claim 1, wherein the occlusion instrument furthermore comprises one or more drugs.

7. Process for producing an occlusion instrument devised for closing defects of the septum in the heart, comprising a yarn of bioresorbable, radiopaque and thermoplastically deformable polymers with or without shape memory characteristics, said polymers comprising radiopaque building groups in the repeat units of the polymer chains, and wherein said occlusion instrument furthermore comprises a bioresorbable fabric, said process comprising solution or melt spinning of the bioresorbable, radiopaque and thermoplastically deformable polymers with or without shape memory characteristics, which on the one hand contain radiopaque triiodophenyl side groups conforming to the formula (I) in the repeat units of the polymer chains to produce at least one thread and subsequently processing said at least one thread by a shaping processes into said occlusion instruments. 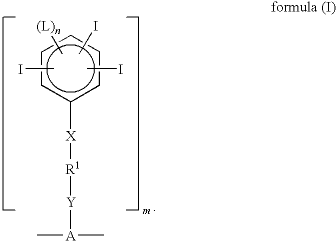
where
L represents H or a moiety comprising a functional group selected from the group consisting of a carboxylic acid, an amine, a phosphate, a phosphonic acid, a sulfate, a sulfonic acid, an oligo(ethylene oxide), an amide, protonated or deprotonated states thereof, and salts thereof,
n=0, 1, or 2,
Y and X are, independently, absent or represent bonding groups,
R¹ is absent or represents an at least 2-valent linear, branched or cycloorganic moiety comprising 1 to 15 carbon atoms,
A represents an m+2-valent linear, branched or cycloorganic moiety containing 1 to 30 carbon atoms, and
m=1, 2, 3, or 4, and
the phenyl moiety is substituted with the iodine atoms in free o-, m- or p-positions.

8. Occlusion instrument obtainable by the process according to claim 7.

9. The occlusion instrument according to claim 1, wherein the bioresorbable, radiopaque and thermoplastically deformable polymers comprise triiodophenyl side groups as the aforementioned radiopaque building groups, conforming to the formula (I): 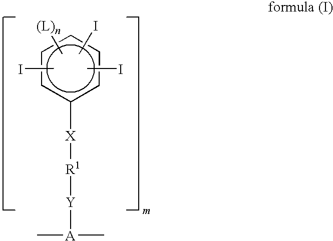
where
L represents H or a moiety comprising a functional group selected from the group consisting of a carboxylic acid, an amine, a phosphate, a phosphonic acid, a sulfate, a sulfonic acid, an oligo(ethylene oxide), an amide, protonated or deprotonated states thereof, and salts thereof,
n=0, 1, or 2,
Y and X are, independently, absent or represent bonding groups,
R¹ is absent or represents an at least 2-valent linear, branched or cycloorganic moiety comprising 1 to 15 carbon atoms,
A represents an m+2-valent linear, branched or cycloorganic moiety containing 1 to 30 carbon atoms, and
m=1, 2, 3, or 4, and
the phenyl moiety is substituted with the iodine atoms in free o-, m- or p-positions.

10. The occlusion instrument according to claim 9, wherein
L represents H or a moiety comprising a functional group selected from the group consisting of a carboxylic acid, an amine, a phosphate, a sulfate, an oligo(ethylene oxide), an amide, protonated or deprotonated states thereof, and salts thereof,
n=0, 1 or 2,
Y and X are, independently, absent or represent bonding groups, such as carboxylic ester or urethane groups,
R¹ is absent or represents an at least 2-valent linear, branched or cycloorganic moiety comprising from 1 to 10 carbon atoms,
A represents an m+2-valent linear, branched or cycloorganic moiety containing 1 to 20 carbon atoms,
m=1 or 2, and
the phenyl moiety is substituted with the iodine atoms in free o-, m- or p-positions.

11. The occlusion instrument according to claim 1, wherein the uncross-linked radiopaque polymers formed after incorporation of the radiopaque building groups in the bioresorbable polymers are crosslinked by diisocyanates.

12. The occlusion instrument according to claim 1, wherein uncross-linked radiopaque polymers formed after incorporation of the radiopaque building groups in the bioresorbable polymers are provided with free-radically polymerizable groups by end group modification and the telechels formed are subsequently copolymerized in the presence of a free-radical initiator and if appropriate one or more free-radically polymerizable co-monomers to form a biodegradable, radiopaque polymer network.

13. The occlusion instrument according to claim 1, wherein the bioresorbable, radiopaque and thermoplastically deformable polymers are admixed, before or during shaping, with additives which effect an adjustment and adaptation of the mechanical thermal properties and/or specific application properties of the polymers.

14-26. (canceled)
